(19) United States
(12) Patent Application Publication
(10) Pub. No.: US 2025/0363992 A1
(43) Pub. Date: Nov. 27, 2025

(54) RADAR MARKER

(71) Applicant: ALTAVO GMBH, Dresden (DE)

(72) Inventors: Martin LAABS, Dresden (DE); Dirk PLETTEMEIER, Dresden (DE)

(21) Appl. No.: 18/874,009

(22) PCT Filed: Jun. 14, 2023
(86) PCT No.: PCT/EP2023/065985

§ 371 Date: Dec. 11, 2024

(30) Foreign Application Priority Data
Jun. 15, 2022 (DE) 10 2022 115 035.6

(51) Int. Cl. G10L 15/24; G01S 7/35; G01S 13/75; G01S 13/88

(52) U.S. Cl. CPC G10L 15/24; G01S 7/354; G01S 13/755; G01S 13/88

(57) ABSTRACT

Techniques for characterizing a vocal tract by using radar measurements. A radar marker that is arranged in or on the vocal tract is used.

### TECHNICAL FIELD

Various aspects relate to a radar marker that can be used to make high-precision localization of structures in the context of a near-body radar measurement possible. Various aspects relate to the execution of a near-body radar measurement using an appropriate radar marker to synthesize a person's speech based on corresponding radar signals.

### BACKGROUND

It is known to use antenna arrangements in near-body applications in order to employ electromagnetic waves within a radar measurement to measure anatomical structures of a person.

One example relates to the characterization of the vocal tract. The characterization of the vocal tract can be done for the purpose of determining an intended speech utterance of the person. See, for example, Wagner, Christoph, et al. “Silent Speech Command Word Recognition Using Stepped Frequency Continuous Wave Radar.” (2021).

Technical descriptions of the human voice often use the so-called source-filter model. The lungs, trachea, and larynx together form the source. The air is compressed in the lungs and flows through the trachea up to the larynx. In the larynx, the vocal folds-colloquially known as “vocal cords”-form the glottis. The larynx muscles keep the vocal folds under tension by exerting force through the arytenoid cartilages. When speaking with a voice, the pressure in the trachea and the tension of the vocal folds cause them to open and close periodically, thus creating an acoustic vibration, a sound wave. This sound wave is acoustically filtered by the time-varying shape of the vocal tract, which consists of the pharynx, oral cavity and nasal cavity, before it exits the mouth and nostrils.

The production of speech from corresponding intended speech utterances consists of the process of phonation, technically speaking the excitation of an acoustic vibration by the vocal folds, and articulation, i.e., the filtering of the sound spectrum by the time-varying shape of the vocal tract. The vocal tract is formed by the soft palate, which opens or closes the nasal cavity, of the tongue, of the upper and lower row of teeth, and of the upper and lower lips.

To determine an intended speech utterance, the vocal tract can be characterized. For example, it may be possible to measure the relative position of the vocal-tract-forming body parts (e.g., tongue, lips, soft palate, etc.) and their movement, and to record them clearly over time.

### BRIEF SUMMARY

It is a task of the invention to provide techniques that allow to characterize structures by means of a near-body radar measurement. In particular, it is the task of the invention to provide techniques for characterizing a person's vocal tract by using radar signals.

This task is solved by the features of the independent claims. The features of the dependent claims define embodiments.

A method for characterizing a person's vocal tract comprises performing a near-body radar measurement. A radar measurement can, for example, comprise the determination of the impulse response of the channel. That means that a radar measurement can comprise an interaction of electromagnetic waves with the tissue and the receipt of information about this interaction. Radar signals are obtained in this way. For this purpose, a radar marker is arranged in or on the person's vocal tract. Thus, the radar marker is a separate, artificial component that does not belong to the vocal tract but can be arranged in or on the vocal tract. The radar marker can be made of plastic and/or metal, for example. The radar marker can be an active or passive electronic component. The radar marker influences electromagnetic waves of the radar measurement. The radar signals can be evaluated to characterize the vocal tract. The radar signals could, for example, be evaluated to determine an intended speech utterance.

The use of such techniques makes it possible to measure the person's vocal tract. A spatial characterization can be executed or exploited. For example, an exact localization of structures of the vocal tract could be executed or exploited. In particular, the features in the radar signal that originate from the vocal tract can be determined by using the radar marker with a resolution that is higher than a native spatial or temporal resolution of the radar measurement (i.e., without using the radar marker). For example, the native resolution of the radar measurement could be limited by the number of antenna elements used in an antenna arrangement and by the bandwidth of the radar signal. A lateral component of the native resolution can describe how large structural distances between neighboring features must be in order to be separated by the radar measurement with sufficient probability. The depth resolution is the accuracy with which distances of the measured structure to the antenna can be measured, typically over the travel time of the electromagnetic waves. Both the lateral resolution and the depth resolution can be improved by using the radar marker. This increased resolution can be achieved selectively in conjunction with the radar marker; i.e., not generally over the entire field of view of the radar measurement for any structure.

Furthermore, it may be possible to improve the signal-to-noise ratio of the received signal by using such techniques. In particular, interferences—for example, electromagnetic waves from the radar measurement that follow paths other than those contacting the vocal tract, or external interference signals—can be better distinguished from signal contributions of the radar signals that contain information about the person's vocal tract. In particular, such techniques make it possible to achieve high signal-to-noise ratios with limited transmitting powers for the electromagnetic waves (typically required due to the near-body radar measurement).

As a general rule, a wide variety of radar marker types can be used in the methods disclosed herein. For example, delay lines could be used to influence electromagnetic waves. See for example: Reindl, Leonhard, et al. “Design, fabrication, and application of precise SAW delay lines used in an FMCW radar system.” IEEE Transactions on microwave theory and techniques 49.4 (2001): 787-794. Another example concerns frequency conversion, see for example Tahir, Nazifa, and Graham Brooker. “Recent developments and recommendations for improving harmonic radar tracking systems.” Proceedings of the 5th European Conference on Antennas and Propagation (EUCAP). IEEE, 2011.

In particular, it is possible in various examples to use an active radar marker. Such a radar marker can change the influence of the electromagnetic waves over time in order to encode information into the electromagnetic waves. This information can be decoded in the form of data from the electromagnetic waves. In this way, further applications can be made possible, for example the separation of the signal contributions from different radar markers used in parallel, or the transmission of additional measurement information that can be used, for example, to determine the intended speech utterance.

An exemplary radar marker comprises an adjustable element that can be switched between different settings. In addition, the active radar marker also comprises a reflector structure. This structure is designed to influence electromagnetic waves of a radar measurement differently depending on the setting of the adjustable element. In such a solution, the active radar marker is designed to change the setting of the adjustable element in order to encode data into the electromagnetic waves or to modulate the signal by the movement of the marker.

The features and characteristics set forth above and those described below may be used not only in the corresponding explicitly explained combinations but also in further combinations or in isolation without departing from the scope of protection of the present invention.

### BRIEF DESCRIPTION OF THE DRAWINGS

FIG. 1 is a flow chart of an exemplary method.

FIG. 2 schematically illustrates a data processing pipeline for evaluating radar signals to synthesize speech according to various examples.

FIG. 3 schematically illustrates an active radar marker according to various examples.

FIG. 4 schematically illustrates a system according to various examples.

### DETAILED DESCRIPTION

The characteristics, features and advantages of the present invention described above, as well as the way in which they are achieved, will become clearer and more precisely understandable in connection with the following description of the exemplary embodiments which will be discussed in more detail with reference to the figures.

In the following, the present invention will be discussed in more detail by means of preferred embodiments with reference to the figures. The same reference numerals in the figures denote the same or similar elements. The figures are schematic representations of various embodiments of the invention. The elements depicted in the figures are not necessarily shown to scale. Rather, the various elements illustrated in the figures are represented in such a way that their function and general purpose are understandable to those skilled in the art. Connections and couplings between functional entities and elements illustrated in the figures can also be implemented as indirect connections or couplings. A connection or coupling may be implemented in a wire-based or wireless fashion. Functional entities may be implemented as a hardware or software solution, or as a combined solution of hardware and software.

Techniques that allow to characterize a person's vocal tract are described below. In the following, techniques are described that allow to determine a person's intended speech utterance. This is made possible by the characterization of the vocal tract. The intended speech utterance could be described by individual sounds or a sequence of sounds. It would be conceivable to determine a text that describes a textual reproduction of the speech. An audio output could be determined (i.e., an audio signal) that corresponds to the intended speech utterance. In the following, the term “determination of a synthetic speech of a person” is used for all such variants.

In particular, synthesized speech can even be determined if the person is unable to produce sounds. This means that “silent speech” is made possible. Voice disorders manifest themselves in a set of symptoms, which are grouped under the term dysphonia: from hoarseness to a weak or distorted speech, to a complete loss of voice, known as aphonia. Voice disorders can have a functional or organic origin. Organic voice disorders can be further subdivided into structural or neurogenic disorders. A “silent speech” can be made possible for persons with such voice disorders.

Synthesized speech can also be generated if the person intentionally whispers or silently articulates. This could, for example, allow a private “conversation” over the phone.

To obtain the required characterization of the vocal tract, a near-body radar measurement is used to measure the person's vocal tract. A radar marker is used to improve the resolution and the signal-to-noise ratio of the radar measurement. The radar marker is arranged in or on the person's vocal tract. The radar marker could also be implanted in the area of the vocal tract. The radar marker could be placed in the tongue area, for example. The radar marker could be arranged on the soft palate, which opens or closes the nasal cavity.

In principle, the radar marker can be designed in different ways. For example, the radar marker could be designed as a reflector. The signal reflected by the radar marker comes as a reflection from one, possibly known, location in contrast to the scattered signal originating, for example, from the tissue. This a-priori information, which can be represented as an approximation of the radar marker as a point source of the reflected signal, supports the subsequent data processing. The reflection properties of the radar marker can be appropriately designed. In this way it can be ensured that several signal contributions of the radar signals can be distinguished from one another or can be separated. For example, signal contributions that encode information about the vocal tract can be separated from signal contributions that correspond to propagation paths of the electromagnetic waves that do not run through the vocal tract. In this way, it can be determined, for example, whether certain features in the radar signals originate from bones or other implants or instruments in the vicinity of the vocal tract, or whether they contain information about the vocal tract.

For such an implementation of the radar marker, a nonlinear influence on the reflected signal could be used, for example. For example, the frequency of the incident electromagnetic waves could be changed such that the reflected electromagnetic waves correspond to a harmonic of the incident electromagnetic waves. Alternatively or additionally, a modulation, such as a periodic modulation, of the reflected electromagnetic waves could be realized. The amplitude can be modulated. In this way, information can be encoded. It would also be conceivable to delay the propagation time of the electromagnetic waves.

In the different scenarios, it is desirable for the radar marker that it requires no or only a comparatively small amount of energy to operate. If energy is required, it can be provided by a suitable energy source. For example, a battery could be used. An energy converter, which takes energy from the electromagnetic field and makes it available for the operation of the radar marker could also be used; for this purpose, a rectifier circuit can be provided that converts high-frequency (RF) currents into direct currents. It would also be conceivable to use inertial structures that provide energy when accelerated, which means, for example, motion-induced charging of a capacitor. This phenomenon is also known as “energy harvesting”.

In various examples, multiple differently positioned radar markers can also be used, for example to distinguish different sub-areas of the vocal tract—for example, palate and tongue—from one another. The different signal contributions associated with the multiple radar markers can then be separated in different ways. For example, if the radar markers are positioned at different distances to the antenna, a separation can be made by using the different propagation times of the electromagnetic waves. Different delays of the propagation time could be imposed. Different conversion factors could be used for a change in frequency. Different/orthogonal sequences could also be used for modulating the amplitude of the electromagnetic waves by the different radar markers.

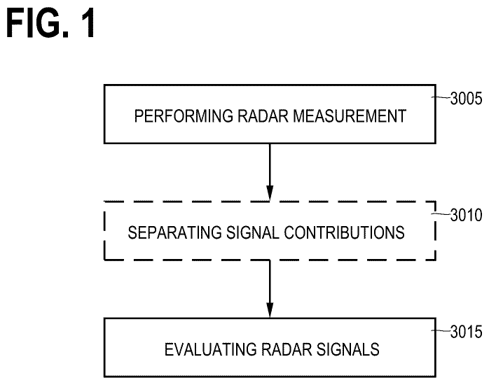

FIG. 1 is a flow chart of an exemplary method.

In Box 3005, a radar measurement is carried out. The radar measurement is carried out close to the body. This means that an antenna can be attached close to a person's body so that the antenna emits electromagnetic waves into the person's body or propagates the electromagnetic waves along the skin surface. For example, an antenna applied to a flexible film could be used and, then, the flexible film can be applied to the surface of the person's skin.

Principally, the radar measurement can be carried out in reflections or transmission. In particular, multiple antennas can be turned, which are arranged on different sides of a structure to be measured—in particular the person's vocal tract. In this way, the transmission of electromagnetic waves through the person's vocal tract can be measured.

Based on the radar measurements, radar signals are obtained. These signals include signal contributions that carry information about the vocal tract (through reflection or transmission at the vocal tract) as well as other signal contributions. The other signal contributions correspond to propagation paths that do not run through the vocal tract.

A radar marker that has an influence on the electromagnetic waves of the radar measurement is arranged at or in the person's vocal tract. The radar marker makes it possible to localize the structures of the vocal tract in a very precise manner. Features in the radar signals that encode information about the vocal tract can be selectively identified. Signal contributions that are associated with the vocal tract or other structures in the vicinity can be separated in this way. Interference can be reduced. The signal-to-noise ratio can be increased. This is particularly useful for separating different speech utterances that are produced by only slightly different configurations of the vocal tract: certain pairs of consonants that are only produced by a slightly different tongue position would be an example.

For example, it would be optionally possible, in Box 3010, that a vocal tract signal contribution of the radar signals is determined. The vocal tract signal contribution can be generated by the interaction of the electromagnetic waves at the radar marker. Such a determination of the vocal tract signal contribution can be based on prior knowledge about the influence of the radar marker on the electromagnetic waves. Then, it is possible to specifically evaluate the vocal tract signal contribution in Box 3015.

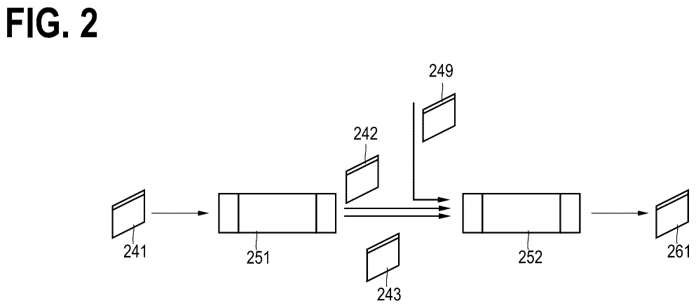

Broadly speaking, the radar signals are evaluated in Box 3015. The evaluation can be used, in particular, to determine a synthetic language of the person. To do this, previously known techniques, such as those described by Wagner, Christoph, et al. “Silent Speech Command Word Recognition Using Stepped Frequency Continuous Wave Radar.” (2021), can be taken as a basis. A modified pipeline for data processing is shown in FIG. 2.

FIG. 2 illustrates aspects concerning radar signal processing in order to characterize a vocal tract. This characterization of the vocal tract is done in conjunction with the determination of an intended speech utterance. A synthetic speech can be determined. FIG. 2 shows a data processing pipeline.

Radar signals 241 are obtained by using a radar measurement close to the body. The radar signals 241 could correspond, for example, to the impulse response of the propagation channel of the radar waves.

In a preprocessing algorithm 251, two signal contributions 242, 243 of the radar signals 241 are determined. The vocal tract signal contribution 242 is generated by the interaction of the electromagnetic waves of the underlying radar measurement at the person's vocal tract. The signal contribution 243 is complementary to the vocal tract signal contribution 242.

Optionally, the preprocessing algorithm 251 could also provide further filtering of the radar signals 241, e.g., remove static background information, etc.

The signal contributions 242, 243 are then input data into an algorithm 252 that provides output data associated with a characterization of the vocal tract. This characterization could be realized, for example, in the form of a localization or in the form of structures of the vocal tract or of a part of the vocal tract; corresponding information could be indicated by the output data 261. However, it is also conceivable that the output data 261 describe the intended speech utterance derived from the characterization of the vocal tract. For example, a sequence of sounds could be indicated. A corresponding audio signal or a text representation could be output.

In principle, it is optional that the signal contribution 243 is also forwarded to the algorithm 252 in the form of the input data.

The algorithm 252 can be, for example, a machine-learned algorithm. An artificial neural network could be used, for example. In particular, a convolutional network could be used, i.e., that comprises one or more convolutional layers in which the corresponding feature maps are convolved with a trained kernel. A recurrent neural network could be used.

The algorithm 252 can be trained, for example, by asking a person to read text aloud (with or without phonation). Based on the text, a target output of the algorithm 252 can be determined. Simultaneously, training radar signals 241 can be determined. Based on previously known training methods, the algorithm 252 can be trained, e.g., by applying a gradient descent optimization method (backward propagation).

In principle, the algorithm 251 can also be machine-learned. In this case, an end-to-end training of the two algorithms 251, 252 could be used. However, the algorithm 251 could also be parameterized manually; this method is explained below.

The algorithm 251 can operate based on prior knowledge of the influence of the radar marker on the electromagnetic waves. For example-depending on the mode of operation of the radar marker works-a corresponding effect could be exploited to separate the signal contributions 242, 243. Some examples are given below in connection with TABLE 1.

TABLE 1
Various examples for determining a signal contribution
which arises from a specific radar marker.

| | FUNCTIONAL | |
|---|---|---|
| | PRINCIPLE | |
| | RADAR | |
| | MARKER | EXEMPLARY DETAILS |
| I | Separation | The radar marker could, for example, change a |
| | int the | frequency of the electromagnetic waves. This |
| | frequency | change can be achieved by nonlinear effects. |
| | domain | Harmonic frequency components can be |
| | | generated, for example, by inserting diodes into |
| | | the RF signal path. In principle, external energy |
| | | supply is not necessary for this process. |
| | | Alternatively, a so-called frequency mixer |
| | | (multiplier) can be used to shift the radar signal in |
| | | the frequency range. By changing the frequency of |
| | | the electromagnetic waves, the signal contribution |
| | | 242, for example, could be determined by means |
| | | of appropriate filtering in the frequency domain. |
| II | Separation | The radar marker could, for example, delay the |
| | in time | propagation time of the electromagnetic waves. |
| | domain | This delay could be achieved, for example, by |
| | | converting the electromagnetic waves into surface |
| | | acoustic waves (SAW) and, then, reconverting |
| | | them into electromagnetic waves. In principle, |
| | | external energy supply is not necessary for this |
| | | process. The acoustic surface waves travel |
| | | comparatively slowly in a transmission path so that |
| | | the transmission time is delayed. Then, the signal |
| | | contribution can be determined by means of |
| | | appropriate filtering in the time domain. |
| III | Separation | It is also conceivable that the radar marker |
| | in code | effectuates a modulation of the electromagnetic |
| | domain | waves of the radar measurement. A reference |
| | | code can be used for the modulation. In this way, a |
| | | separation can be made in the code domain, if the |
| | | reference code is known. For example, certain |
| | | code sequences, such as a pseudo-random noise |
| | | code sequence, can be used. |

The techniques listed in TABLE 1 can be used to determine the signal contribution for a defined radar marker, see FIG. 2, signal contribution 242.

The techniques described in TABLE 1 can be used to separate also signal contributions for several differently positioned radar markers. For this purpose, it is conceivable, for example, that the operating parameters of the various radar markers are different. For example, a first radar marker could cause a frequency conversion by a first factor and a second radar marker could cause a frequency conversion by a second factor that is different from the first factor, see Example I. Different delays for the electromagnetic waves could also be used, see Example II. Orthogonal codes could be used for the modulation, see Example III.

Various variations of the data processing pipeline according to the example given in FIG. 2 are conceivable. For example, in various examples, it is conceivable that there a discrete separation of the contributions 242, 243 is not implemented in the preliminary stages of the machine-learned algorithm 252. In other words, it is conceivable that the machine-learned algorithm 252 directly receives the radar signals 241 as an input. Based on suitable training data (which, for example, correlate speech provided by the user with training radar signals), it is then conceivable that a separation of the different signal contributions or an extraction of the relevant features, which are associated with the vocal tract, is provided in the machine-learned algorithm 252.

Another possible variation of the data processing pipeline according to the example of FIG. 2 relates to the expansion of the input data for the machine-learned algorithm 252. The example of FIG. 2 shows that the input to the machine-learned algorithm 252 also includes further data 249 (generally optional). For example, it is conceivable that the further data 249 are recorded by using a further measuring method. For example, such data 249 could be obtained in ultrasound measurement methods that measures the person's vocal tract using other sensor systems. A camera could be used to measure the movement of the skin surface in the area of the vocal tract. Alternatively or in addition to using a different measurement method, it is also conceivable to extract the data 249 from the radar signals 241 (not shown in FIG. 2). In particular, it is conceivable that, based on prior knowledge about the influence of the radar marker on the electromagnetic waves, the radar signals are evaluated in order to determine the data sent by the radar marker. Then, the vocal tract can be characterized based on these data sent by the radar marker.

Depending on the influence of the radar marker on the electromagnetic waves (see TABLE 1), these data can be encoded differently by the electromagnetic waves. For example, it would be possible to encode information by adjusting the frequency conversion and/or the delay of the travel time (cf. TABLE 1: Example I and Example II) depending on the data to be sent. However, it is also conceivable that the data are encoded by a selection of the corresponding code sequences (cf. Table 1: Example III). A data sequence could be directly modulated. Furthermore, it is conceivable, for example, that the change in the amplitude of the reflected electromagnetic waves at the radar marker encodes corresponding data.

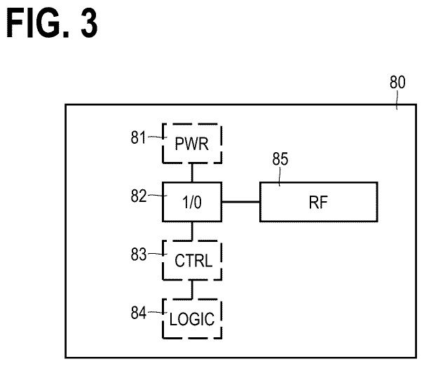

There are different variants for the information encoded by such data transmitted by the radar marker. For example, it is conceivable that such data is indicative of a distance between the radar marker and a predetermined anatomical feature of the person. In this way, it is conceivable, for example, that the relative positioning of the vocal tract is encoded. The data could also be, alternatively or additionally, indicative of an acceleration of the radar marker. In order to provide such and other data, the radar marker can comprise a corresponding passive or active sensor that causes an influence on the interaction of the radar marker with the electromagnetic waves to encode the data. Such and further details in connection with the radar marker are described below in connection with FIG. 3.

FIG. 3 illustrates aspects related to an active radar marker 80 according to various examples.

The active radar marker 80 comprises a reflector structure 85. This structure can influence incoming electromagnetic waves, that means in particular, for example, reflect and/or deflect them. An attenuation could be imposed, a propagation time could be extended and/or the frequency could be converted (cf. TABLE 1). For this purpose, the reflector structure 85 could, for example, comprise a delay line for electromagnetic waves or a frequency converter structure for electromagnetic waves.

The radar marker is designed as an active component, i.e., it modifies one or more properties of the influence of the electromagnetic waves in a time-varying manner. For this purpose, the active radar marker 80 also comprises an adjustable element 82, which can be switched between different settings. Depending on the setting of the adjustable element 82, the electromagnetic waves of the radar measurement are influenced differently by the reflector structure 85.

In one example, the setting of the adjustable element could be selected such that several radar markers can be operated in parallel. The corresponding signal contributions can be distinguished by appropriately selecting the setting of the adjustable element. In another example, the setting of the adjustable element 82 is changed as a function of time in order to encode data into the electromagnetic waves.

The adjustable element 82 can be implemented in different variants. These variations are summarized in TABLE 2.

TABLE 2
Various examples of an implementation of an adjustable element

| | EXAMPLE | BRIEF DESCRIPTION |
|---|---|---|
| I | Active | For example, the adjustable element could be a switch |
| | switch | element that can be switched between different discrete |
| | | switch positions, e.g., on/off. A transistor could be used. |
| | | In order to make such switching of the switch element |
| | | possible, the radar marker 80 can also comprise a |
| | | corresponding energy source (PWR) 81. This energy |
| | | source 81 is designed to provide energy for switching |
| | | the switch element (1/0) 82 between the at least two |
| | | switch positions. In the example of FIG. 3, the radar |
| | | marker also comprises a logic element (LOGIC) 84. This |
| | | element is designed to provide control data. Then, a |
| | | driver circuit (CTRL) 83 is configured to switch the |
| | | switch element 82 between at least two switch positions |
| | | based on this control data. |
| | | For example, the logic element 84 may comprise a non- |
| | | volatile memory. This memory may be configured to |
| | | store at least a portion of the control data. Such a |
| | | scenario will be conceivable, for example, if the control |
| | | data encodes an identity of the radar marker 80; for |
| | | example, in order to achieve a separation of different |
| | | signal contributions for different radar markers based on |
| | | orthogonal codes (cf. Tab. 1: Example III). |
| | | It is also conceivable that the logic element 84 |
| | | comprises a sensor. This sensor can be configured to |
| | | determine at least a portion of the control data based on |
| | | a measurement. For example, a temperature could be |
| | | measured locally. It is conceivable, for example, that an |
| | | acceleration is measured. Then, corresponding |
| | | measured values of the control data can be provided for |
| | | switching the switch element 82 in order to encode the |
| | | data into the electromagnetic waves in this way. |
| | | For example, the driver circuit 83 could be configured to |
| | | switch the adjustable element at a rate of at least |
| | | 500 Hz, optionally at least at 1 KHz. This means that |
| | | switching can be realized multiple times between the |
| | | duration of a radar chirp, which typically has a duration |
| | | of <10 ms. |
| | | The energy source can comprise a rectifier circuit. The |
| | | rectifier circuit can be configured to provide a direct |
| | | current. The rectifier circuit can provide the direct |
| | | current based on the electromagnetic waves or another |
| | | low-frequency electromagnetic field. For this purpose, |
| | | the energy source could also be coupled to the reflector |
| | | structure 85. The energy source 81 could also have an |
| | | inertial structure for motion-induced charging; an energy |
| | | store could be provided for this purpose. |
| II | Variable | It is also conceivable that the reflector structure 85, |
| | oscillating | together with the adjustable element 82, implements an |
| | circuit | RF oscillating circuit. The adjustable element 82 can, for |
| | | example, be a variable capacitance. The capacitance |
| | | value of the variable capacitance can then change |
| | | depending on the distance of the active radar marker to |
| | | a counter-electrode formed by the tissue or an artificial |
| | | counter-electrode. For example, the counter-electrode |
| | | designed as a metallic layer could be glued to a skin |
| | | surface near the vocal tract. In this way, the distance to |
| | | this counter-electrode could be detected in the manner |
| | | of a distance sensor. Then, the change in the |
| | | capacitance value leads to a change in the resonant |
| | | frequency of the RF oscillating circuit, thus continuously |
| | | changing the attenuation or frequency response of the |
| | | electromagnetic waves. This corresponds to a variable |
| | | attenuation or frequency response that encodes the |
| | | distance values. An energy source, a driver circuit and a |
| | | logic element are not required in this implementation; |
| | | therefore, these elements are shown in FIG. 3 in dashed |
| | | lines. |
| | | If auxiliary power is available, a variable oscillator (e.g., |
| | | at 10 kHz), the frequency of which switches the antenna |
| | | switch, could also be used. Then, the frequency of the |
| | | oscillator is changed by a variable capacitance. The |
| | | capacitance value of the variable capacitance depends |
| | | on the distance between a corresponding electrode and |
| | | a counter-electrode. Thus, it is not necessary to |
| | | modulate directly onto the carrier frequency of the |
| | | electromagnetic waves. |

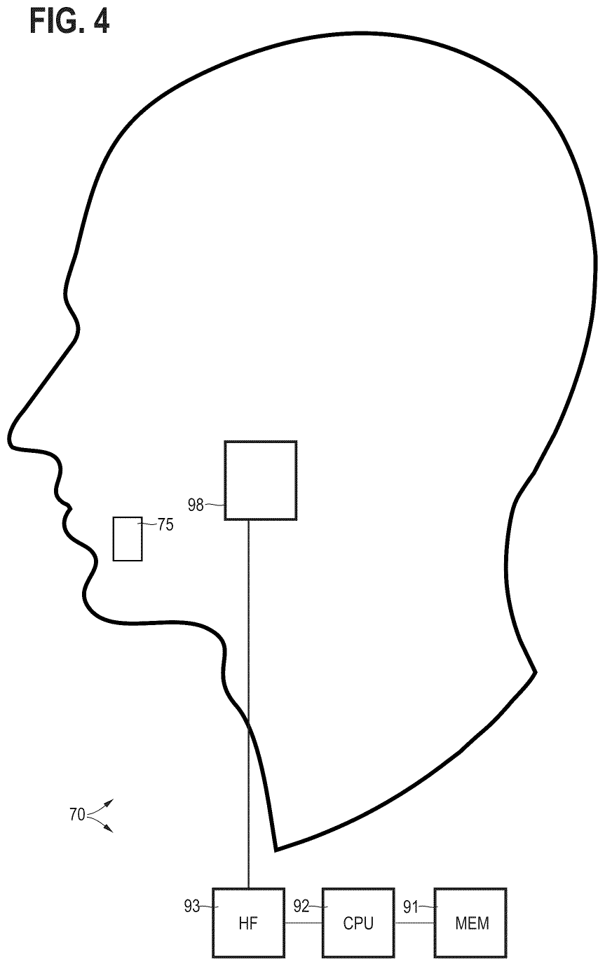

FIG. 4 illustrates an exemplary system 70. The system 70 comprises an RF front end 93 coupled to an antenna 98. The RF front end 93 can couple RF signals into the antenna 98 in order to emit electromagnetic waves. Alternatively or additionally, it is also conceivable that the RF front end 93 receives radar signals from the antenna 98.

The RF front end can, for example, comprise a frequency mixer, an oscillator, an RF amplifier and a filter.

In the example of FIG. 4, the system 70 comprises only a single antenna 98. However, in general it is conceivable—in particular for a transmission measurement—that the RF front end 93 is coupled to both a transmitting antenna and a receiving antenna.

FIG. 4 also shows that a radar marker 75 is arranged in or on a person's vocal tract, for example the radar marker 80 according to the example of FIG. 3 or another radar marker. For example, a passive radar marker could be used that has a non-adjustable reflector structure that is configured to delay electromagnetic waves of the radar measurement and/or to change a frequency of the electromagnetic waves. A radar marker as described in US 2021/0068705 A1 could be used.

Several radar markers that are arranged offset from one another could also be used.

The radar marker 75 could be attached, for example, to the person's tongue.

The RF front end 93 (as a receiver) can provide radar signals 241, which are then processed by a processor 92 based on a program code from a memory 91. For example, data could be processed according to the pipeline shown in FIG. 2. In particular, the processor 92 can be configured to characterize the person's vocal tract based on the program code, for example in order to synthesize a speech of the person.

Of course, the features of the embodiments and aspects of the present invention described above can be combined with one another. In particular, the features can be used not only in the combinations described but also in other combinations or on alone their own without departing from the field of this invention.

For example, techniques have been described above for using a radar measurement close to the body with the aid of a radar marker in order to characterize the vocal tract and to determine an intended speech utterance of the person. However, other application scenarios for such a near-body radar measurement using a radar marker as disclosed above is also conceivable. For example, it would be possible to localize the tip of a catheter or its position in the body. Thus, it would be possible to realize the localization using an alternative method, for example a method based on X-ray images. The position of the tip of the catheter could be registered with a preoperative volume image data set, such as a magnetic resonance image data set and a computer tomography image data set. Instead of a catheter, other instruments can also be localized accordingly. Another application would be, for example, the marking of implants, for example, to detect the loosening of an implant at an early stage. Certain capsules or elements can be marked, such as the camera capsule in a colonoscopy.

Furthermore, aspects related to active radar markers have been described. However, passive radar markers could also be used. For example, purely passive contrast-enhancing scatterers could be used as radar markers. Examples would be piercings or scatterers or gold foils implemented on the skin or subcutaneously, etc. In summary, various types of artificial radar markers can be used, which, for example, can be implanted in the vocal tract or attached onto the vocal tract.

1. A method for characterizing a person's vocal tract, comprising:
performing a near-body radar measurement to obtain radar signals, wherein a radar marker that influences electromagnetic waves of the radar measurement is arranged in or on the person's vocal tract, and
evaluating the radar signals to characterize the person's vocal tract.

2. The method according to claim 1, the method further comprising:
based on prior knowledge about an influence of the radar marker on the electromagnetic waves: determining a vocal tract signal contribution of the radar signals, which is generated by the interaction of the electromagnetic waves at the radar marker,
wherein the vocal tract signal contribution of the radar signals is evaluated to characterize the vocal tract.

3. The method according to claim 2,
wherein input data of a machine-learned algorithm includes the vocal tract signal contribution,
wherein the machine-learned algorithm provides output data that characterize the vocal tract.

4. The method according to claim 3, the method further comprising:
determining a complementary signal contribution of the radar signals that is complementary to the vocal tract signal contribution,
wherein the input data of the machine-learned algorithm further includes the complementary signal contribution.

5. The method according to claim 2, the method further comprising:
based on further prior knowledge about a further influence of a further radar marker, arranged at a distance from the radar marker, on the electromagnetic waves: determining a further signal contribution of the radar signals, which is generated by the interaction of the electromagnetic waves at the further radar marker,
wherein the further signal contribution of the radar signals is evaluated to characterize the vocal tract.

6. The method according to claim 1,
wherein input data of a machine-learned algorithm comprises the radar signals,
wherein the machine-learned algorithm provides output data that characterize the vocal tract.

7. The method according to claim 1,
wherein the radar marker delays a propagation time of the electromagnetic waves of the radar measurements.

8. The method according to claim 1,
wherein the radar marker changes a frequency of the electromagnetic waves of the radar measurement.

9. The method according to claim 1,
wherein the radar marker effects a modulation of the electromagnetic waves of the radar measurement.

10. The method according to claim 1, the method further comprising:
based on prior knowledge about the influence of the radar marker on the electromagnetic waves: evaluating the radar signals to determine data that are sent by the radar marker,
wherein the data sent by the radar marker are evaluated to characterize the vocal tract.

11. The method according to claim 10,
wherein the data are indicative of a distance of the radar marker to a predetermined anatomical feature of the person.

12. The method according to claim 10,
wherein the data are indicative of an acceleration of the radar marker.

13. An active radar marker for an in-body application, comprising:
an adjustable element that can be switched between different settings, and
a reflector structure that is configured to influence electromagnetic waves of a radar measurement differently depending on the setting of the adjustable element,
wherein the active radar marker is configured to change the setting of the adjustable element to encode data into the electromagnetic waves in this way.

14. The active radar marker according to claim 13,
wherein the adjustable element is a switch element that can be switched between at least two switch positions,
wherein the active radar marker further comprises:
an energy source that is designed to provide energy for switching the switch element between the at least two switch positions,
a logic element that is designed to provide control data, and
a driver circuit that is configured to switch the switch element between the at least two switch positions based on the control data.

15. The active radar marker according to claim 14,
wherein the logic element comprises a non-volatile memory that is configured to store at least a portion of the control data.

16. The active radar marker according to claim 14,
wherein the logic element comprises a sensor that is configured to determine at least a portion of the control data based on a measurement.

17. The active radar marker according to claim 14,
wherein the driver circuit is configured to switch the adjustable element at least at 500 Hz.

18. The active radar marker according to claim 14,
wherein the energy source comprises a rectifier circuit which is configured to provide a direct current based on electromagnetic waves.

19. The active radar marker according to claim 14,
wherein the energy source comprises an inertial structure for motion-induced charging.

20. The active radar marker according to claim 13,
wherein the reflector structure together with the adjustable element implements a high-frequency oscillating circuit,
wherein the adjustable element is a variable capacitance that is configured to change its capacitance value depending on the distance of the active radar marker to a counter-electrode.

21. The active radar marker according to claim 13,
wherein the adjustable element is a switch element that can be switched between at least two switch positions,
wherein the active radar marker further comprises:
an energy source that is designed to provide energy for switching the switch element between the at least two switch positions, and
a variable oscillator having a variable capacitance,
wherein the variable oscillator is configured to switch the switch element between the at least two switch positions at a variable switching frequency that is determined in dependence on a capacitance value of the variable capacitance,
wherein the capacitance value of the variable capacitance depends on a distance between an electrode of the variable capacitance and a counter-electrode.

22. The active radar marker according to claim 13,
wherein the reflector structure comprises a delay line for the electromagnetic waves.

23. The active radar marker according to claim 13,
wherein the reflector structure comprises a frequency converter structure for the electromagnetic waves.

24. The active radar marker according to claim 13, further comprising a logic element configured to:
perform a near-body radar measurement to obtain radar signals, wherein the radar marker that influences electromagnetic waves of the radar measurement is arranged in or on a person's vocal tract, and
evaluate the radar signals to characterize the person's vocal tract.
